(19) United States
(12) Patent Application Publication
(10) Pub. No.: US 2012/0123275 A1
(43) Pub. Date: May 17, 2012

(54) INFRARED IMAGING OF CUTANEOUS MELANOMA

(75) Inventor: Jean-Paul Ortonne, Nice (FR)

(73) Assignee: Galderma Research & Development, Biot (FR)

(21) Appl. No.: 13/386,668

(22) PCT Filed: Jul. 23, 2010
(86) PCT No.: PCT/EP2010/060680

§ 371 Date: Jan. 23, 2012

Related U.S. Application Data
(60) Provisional application No. 61/213,886, filed on Jul. 24, 2009.

(51) Int. Cl. A61B 6/00

(52) U.S. Cl. 600/473

(57) ABSTRACT

A process is described for identifying and classifying cutaneous melanoma of a lesion on the skin of a subject by a FTIR spectrometer coupled with a micro-imaging system.

### BACKGROUND OF THE INVENTION

Malignant melanoma accounts for less than 5% of the reported skin cancers but it is the most aggressive form of skin cancer as its incidence and mortality are on the rise (ACS. “Cancer Facts and Figures” 2007, Atlanta: American Cancer Society, 2007). The ABCDE mnemonic (Asymmetry, Border, Colour, Diameter, and Evolution) can aid in the clinical diagnosis, but the ultimate diagnosis of melanoma is based upon the histological evaluation of the lesion. (Marghoob AA, Scope A. “the complexity of diagnosing melanoma”, J Invest Dermatol 2009;129;11-13). As the morphological interpretation is somewhat subjective, especially for intraepidermal lesions, some discrepancies in the diagnosis have been reported and it relies consequently on the expertise of the dermatopathologist. (Urso C, Rongioletti F, Innocenzi D et al., “histological features used in the diagnosis of melanoma are frequently found in benign melanocytic naevi”, J Clin Pathol 2005;58;409-412; Urso C, Rongioletti F, Innocenzi D et al., “interobserver reproducibility of histological features in cutaneous malignant melanoma”, J Clin Pathol 2005;58;1194-1198; Farmer E R, Gonin R, Hanna M P, “discordance in the histopathologic diagnosis of melanoma and melanocytic nevi between expert pathologists”, Hum Pathol 1996;27;528-531; and Glusac E J, “under the microscope: doctors, lawyers, and melanocytic neoplasms”, J Cutan Pathol 2003;30;287-293). Much efforts dedicated to the development of objective automatic image analysis softwares have been reported but as the structures in histological sections are usually complex, the results have not been conclusive yet. (Gerger A, Smolle J. Diagnostic imaging of melanocytic skin tumors. J Cutan Pathol 2003; 30; 247-252).

Fourier Transform Infrared (FTIR) spectroscopy appears as a method of choice to tackle this limitation. Infrared (IR) spectra probe intrinsic molecular composition and interactions which are characteristic of the histopathological state of the tissue; they can be considered as real tissue-specific spectroscopic fingerprints. Considering the recent developments in the spectroscopic systems, tissue sections can be scanned in two dimensions to record spectral images. Contrary to conventional histological hematoxylin-and-eosin (HE) staining, spectral imaging can be performed directly on archival fixed and paraffin-embedded tissues without staining, avoiding also possible alterations of the tissue induced by chemical dewaxing. (Tfayli A, Piot O, Durlach A, Bernard P, Manfait M., “discriminating nevus and melanoma on paraffin-embedded skin biopsies using FTIR microspectroscopy”, Biochim Biophys Acta 2005;1724;262-269; Ly E, Piot O, Wolthuis R, Durlach A, Bernard P, Manfait M., “combination of FTIR spectral imaging and chemometrics for tumour detection from paraffin-embedded biopsies. Analyst” 2008;133;197-205; Wolthuis R, Travo A, Nicolet C et al., “IR spectral imaging for histopathological characterization of xenografted human colon carcinomas”, Anal Chem 2008;80;8461-8469; and Ly E, Piot O, Durlach A, Bernard P, Manfait M., “differential diagnosis of cutaneous carcinomas by infrared spectral micro-imaging combined with pattern recognition”, Analyst 2009;DOI: 10.1039/B820998G). As for skin lesions, some reports have shown that it is possible to diagnose melanoma from normal epidermis or to discriminate between melanoma and benign naevi on the basis of the IR markers specific of the tissue type. (Mordechai S, Sahu R K, Hammody Z et al. “possible common biomarkers from FTIR microspectroscopy of cervical cancer and melanoma”, J Microsc 2004;215;86-91 and Hammody Z, Argov S, Sahu R K, Cagnano E, Moreh R, Mordechai S., “distinction of malignant melanoma and epidermis using IR micro-spectroscopy and statistical methods”, Analyst 2008;133;372-378). To date, no spectral studies rely on the possibility to distinguish between the different types of melanoma, neither to access prognostic markers relative to patient outcome. Yet, the main challenge now is to screen patients as early as possible as the prognosis of melanoma is closely associated with the disease stage at the time of diagnosis. Therefore, various methods facilitating its diagnosis and its treatment are necessary to address unmet needs.

The present invention enhances the FTIR micro-imaging for characterizing various melanoma types to enable the correlation between IR spectral markers in primary melanoma and some dermatopathological parameters recognized as powerful prognostic factors.

### SUMMARY OF THE INVENTION

The present invention relates to a process for identifying and classifying cutaneous melanoma. The process includes: a) scanning a lesion on the skin of a subject by a FTIR spectrometer coupled with a micro-imaging system; b) acquiring and storing infrared spectra of a series of digital images of the lesion; c) clustering the infrared spectra by a statistics based clustering algorithm, such as a multivariate statistical analyzer using a K-means classification algorithm; d) comparing the cluster-membership information to a spectral library of various tissue types of cutaneous melanoma to identify spectral markers of each tissue type of the cutaneous melanoma; and e) mapping the spectral markers by assigning a color to each different cluster.

In one embodiment, the infrared spectra of the lesion, according to the process of the present invention are real tissue-specific spectroscopic fingerprints.

In another embodiment, the color-coded mapping of the present invention highlights tissue architecture without staining.

The process of the present invention discriminates tumor areas from normal epidermis. Furthermore, the process of the present process differentiates histological subtypes of the cutaneous melanoma.

According to the present invention, each digital image consists of from about 10,000 to about 100,000 spectra. Preferably, each digital image consists of about 30,000 spectra. In addition, each spectrum contains from 1,000 to 50,000 values of absorbance.

Furthermore, each absorbance, according to the present invention is from 720 to 4000 cm⁻¹. Preferably, each absorbance is from 900 to 1800 cm⁻¹.

In yet another embodiment, the centers of the K-means cluster are normalized across the entire skinned area and a second-order derivative is calculated using Savitsky-Golay smoothing on the normalized spectra.

According to the present invention, the process identifies different types of stroma reactions by clustering the infrared spectra. Additionally, Peritumoral stroma and intratumoral stroma are distinguished from the normal stroma reaction.

### DESCRIPTION OF FIGURES

FIG. 1: Spectral histology obtained from a superficial spreading melanoma. A, K-means color-coded image built with 11 clusters, scale bar: 200 μm. B, Dendrogram obtained using HCA on the K-means cluster centers. C, HE stained image of the framed area in panel A, scale bar: 5 μm.

FIG. 2: Spectral histology obtained from an ulcerated nodular melanoma. A, K-means color-coded image built with 11 clusters, scale bar: 200 μm. B, Dendrogram obtained using HCA on the K-means cluster centers. C, HE stained images of the framed areas in panel A, scale bar: 5 μm.

FIG. 3: Spectral histology obtained from an invasive melanoma on naevus. A, K-means color-coded image built with 11 clusters, scale bar: 200 μm. B, Dendrogram obtained using HCA on the K-means cluster centers. C, HE stained image of the framed areas in panel A, scale bar: 5 μm.

FIG. 4: Second-order derivatives from spindle (blue) and epithelioid (red) melanoma cells. The mean (bold) ±standard deviation spectra are shown in A, the 920-1110 cm⁻¹ region, and B, in the 1120-1320 cm⁻¹ region.

### DETAILED DESCRIPTION OF THE INVENTION

The present invention provides a process for identifying and classifying cutaneous melanoma. The process includes scanning a lesion on the skin of a subject by a FTIR spectrometer coupled with a micro-imaging system and acquiring and storing infrared spectra of a series of digital images of the lesion According to the present invention, Infrared transmission spectral images can be collected with an imaging system coupled to a FTIR spectrometer. Optionally, the device can be further equipped with a nitrogen-cooled mercury cadmium detector for imaging and a computer-controlled stage. Prior to acquisition, a visible image of the sample may be recorded and the area of interest can be selected by comparison to the corresponding HE stained sections.

Spectral images can be recorded with each digital image consists of from about 10,000 to about 100,000 spectra. Preferably, each digital image consists of about 30,000 spectra. In addition, each spectrum contains from 1,000 to 50,000 values of absorbance.

Furthermore, each absorbance, according to the present invention is from 720 to 4000 cm⁻¹. Preferably, each absorbance is from 900 to 1800 cm⁻¹.

The process of present invention also comprises clustering the infrared spectra by a statistics based clustering algorithm, such as a multivariate statistical analyzer using a K-means classification algorithm.

For example, for each image, K-means clustering can be used to regroup spectra that show similar spectral characteristics, hence similar biomolecular properties. This classification method was described by Lasch P et al. in “imaging of colorectal adenocarcinoma using FT-IR microspectroscopy and cluster analysis”, Biochim Biophys Acta 2004; 1688; 176-186), which is herein incorporated by reference in its entirety. K-means maps may be calculated several times to make sure a stable solution was reached.

The process of present invention further comprises comparing the cluster-membership information to a spectral library of various tissue types of cutaneous melanoma to identify spectral markers of each tissue type of the cutaneous melanoma; and mapping the spectral markers by assigning a color to each different cluster.

The cluster-membership information can be plotted as a colour-coded map by assigning a colour to each different cluster. Each colour-coded map can be provided to the collaborating pathologist for a microscopic comparison with the corresponding HE stained sections. In one embodiment, the color-coded mapping of the present invention highlights tissue architecture without staining.

K-means cluster centers can be normalized across the whole protein content (Amide I and Amide II vibrations) and second-order derivatives calculated using Savitsky-Golay smoothing (15 points, second order) on normalized spectra. Savitsky-Golay smoothing is fully described in “Smoothing and Differentiation of Data by Simplified Least Squares Procedures”, Analytical Chemistry 1964;36;1627-1639, which is herein incorporated by reference in its entirety

In one embodiment, the infrared spectra of the lesion are real tissue-specific spectroscopic fingerprints. As a result, the process of the present invention discriminates tumor areas from normal epidermis. Furthermore, the process of the present process differentiates histological subtypes of the cutaneous melanoma.

### EXAMPLES

### Materials and Methods

### Patients

Ten patients affected with one of the four subtypes of primary cutaneous melanoma, namely superficial spreading melanoma (4 cases), nodular melanoma (3 cases), acral-lentiginous melanoma (1 case) and melanoma on naevus (2 cases) were analyzed in this study. One sample of benign naevus and one of subcutaneous melanoma metastasis were also included. Of these patients examined, 5 were men and 5 women, aged 36 from 82 years. The cases were classified according to the evidence-based staging system for cutaneous melanoma, published by the American joint Committee on Cancer (AJCC) Melanoma Staging Committee (Table 1). Four cases corresponded to melanoma of thickness<1mm and in this category of depth no case presented ulceration; 5 cases had a thickness of 1-4 mm, including 2 cases with ulceration; and one case had a thickness>4 mm with ulceration.

TABLE 1
Histological and clinical information on primary cutaneous melanoma patients

| | | | | | | | Breslow | | | | |
|---|---|---|---|---|---|---|---|---|---|---|---|
| | | | Tumor | Histological | | Clark's | thickness | Cellular | Mitoses/ | | |
| N° | Sex | Age | site | type | Ulceration | level | (mm) | type | mm² | TNM | Stage |
| 1 | M | 82 | Shoulder | NM | yes | 4 | 2 | Epit | >20 | | |
| | | | | | | | | Spin | | | |
| 2 | M | 55 | Back | SSM | yes | 4 | 2 | Epit | 2 | | |
| 3 | F | 47 | Leg | M on naevus | no | 4 | 1.45 | Epit | 7 | | |
| 4 | F | 36 | Scapula | SSM | no | 2 | 0.48 | Epit | 0 | | |
| 5 | M | 41 | Back | M on naevus | no | 2 | 0.35 | Epit | 0 | | |
| 6 | M | 61 | Scalp | SSM | no | 1 | 0 | Epit | 0 | | |
| 7 | F | 65 | Arm | SSM | no | 3 | 0.76 | Epit | 1 | | |
| 8 | F | 44 | Ear | NM | no | 4 | 1.45 | Epit | 2 | | |
| 9 | M | 61 | Back | NM | yes | 4 | 7.5 | Epit | 6 | | |
| | | | | | | | | Spin | | | |
| 10 | F | 39 | Foot | ALM | no | 4 | 1.92 | Epit | 4 | | |
| 11 | | | | BN | | / | / | | | | |
| 12 | F | 74 | | Metastasis | no | / | / | Epit | 2 | | |

Patient Sex: M = male, F = female
NM = nodular melanoma, SSM = superficial spreading melanoma, ALM = acrallentiginous melanoma
Epit: epithelioid cells, Spin: spindle cells
TNM: tumor, nodal metastasis, and metastasis (AJCC: American joint Committee on Cancer)

### Sample Preparation

Tissue specimens were fixed in 10% buffered formalin and paraffin-embedded. For each sample, three ten micron-thick serial sections were cut. The first and last sections were deposited on a glass slide for hematoxylin and eosin (HE) staining and histological diagnosis by the pathologist. The second section was mounted on a calcium fluoride (CaF₂) window (Crystran Ltd., Dorset, UK) for spectral acquisition without further treatment. The section was fixed with a drop of distilled water and the CaF₂ window was subsequently placed onto a slide warmer until complete water evaporation for paraffin to melt and adhere to the window. A paraffin section was also included to be used as a reference for the infrared signal of paraffin.

### Dermatopathological Parameters

Seven dermatopathological parameters were evaluated, corresponding to ulceration, Breslow thickness, Clark's level of invasion, mitoses, regression, cellular type, presence of naevus.

### FTIR Data Collection

Infrared transmission spectral images were collected with the Spectrum Spotlight 300 imaging system coupled to a Spectrum One FTIR spectrometer (both from Perkin Elmer, Courtaboeuf, France) using the image mode. The device is equipped with a nitrogen-cooled mercury cadmium telluride 16-pixel-line detector for imaging and a computer-controlled stage. Prior to acquisition, a visible image of the sample was recorded and the area of interest was selected by comparison to the corresponding HE stained sections. Spectral images were recorded at 8 accumulations with a resolution of 2 cm⁻¹(1 cm⁻¹ data point interval). Each image pixel sampled a 6.25 μm×6.25 μm area at the sample section, permitting the recording of detailed tissular structures. A background spectrum was collected (240 accumulations, 2 cm⁻¹ resolution) on the CaF₂ window to ratio against the single beam spectra. The microscope was isolated in a venting Plexiglas housing to enable purging with dry air and to eliminate atmospheric interferences. Each spectral image consisted in about 30 thousands of spectra, each containing 3282 values of absorbance, spanning the spectral range of 720-4000 cm⁻¹.

### Data Processing

Spectral images were corrected from the contribution of atmospheric water vapour and CO₂ absorption bands by a built-in function of the Perkin Elmer Spotlight software. All further data processing was carried out directly on spectral images using programs written in Matlab 7.2 (The Mathworks, Natick, Mass.) supplied with the PLS toolbox 2.0 (Eigenvector Research Inc., Manson, Wash.). Spectra were analyzed in the fingerprint region (900-1800 cm⁻¹) which has been proved to be the most informative for the IR analysis of biological samples.

### Removal of Paraffin Contribution

The paraffin-embedded samples have been analyzed directly without any prior treatment. To correct for the contribution of paraffin in FTIR spectra, we have developed an automated processing method based on Extended Multiplicative Signal Correction. This method has been successfully applied on skin and colon cancer samples. The detailed description of the method can be found in the following publications, which are herein incorporated by reference in their entirety: Ly E, Piot O, Durlach A, Bernard P, Manfait M., “differential diagnosis of cutaneous carcinomas by infrared spectral micro-imaging combined with pattern recognition” Analyst 2009;DOI: 10.1039/B820998G; Ly E, Piot O, Wolthuis R, Durlach A, Bernard P, Manfait M., “combination of FTIR spectral imaging and chemometrics for tumour detection from paraffin-embedded biopsies”, Analyst 2008;133;197-205; and Wolthuis R, Travo A, Nicolet C., “IR spectral imaging for histopathological characterization of xenografted human colon carcinomas”, Anal Chem 2008;80;8461-8469. For this correction, the number of principal components of paraffin was set to 9 and the order of the polynomial to 4. For each image, outlier spectra were determined by plotting the fit coefficient and the residue.

### K-means Clustering

For each image, K-means clustering was used to regroup spectra that show similar spectral characteristics, hence similar biomolecular properties. This classification method has already proved its potential for the processing of IR data of cancerous tissues. (See Lasch P, Haensch W, Naumann D, Diem M., “imaging of colorectal adenocarcinoma using FT-IR microspectroscopy and cluster analysis”, Biochim Biophys Acta 2004; 1688; 176-186). It is considered as unsupervised except for the number of clusters determined by the operator. K-means maps were calculated several times to make sure a stable solution was reached. The percentage of convergence was set to 99.99% and the number of clusters was set to 11, which appeared to match the histology of the cutaneous tissues analyzed in this study and in previous ones. The cluster-membership information was then plotted as a colour-coded map by assigning a colour to each different cluster. Each colour-coded map was then provided to the collaborating pathologist for a microscopic comparison with the corresponding HE stained sections.

### Unsupervised Hierarchical Clustering Analysis (HCA)

For each image, HCA was performed on the 11 cluster centres (average spectra of the 11 K-means clusters) in order to quantify the spectral distances between the K-means cluster centres. HCA regroups spectra into groups on a minimal distance criterion. The result of such a clustering is displayed in a tree-like diagram called a dendrogram. Neighbouring spectra are grouped into a same group. The distance between the groups formed gives an estimation of their spectral differences. K-means cluster centres that belong to the same group in the dendrogram describe therefore similar biomolecular composition. Euclidian distances and HCA clustering using Ward's algorithm were calculated using built-in Matlab functions from the Statistical toolbox.

### Derivatization

K-means cluster centres were normalized across the whole protein content (Amide I and Amide II vibrations) and second-order derivatives calculated using Savitsky-Golay smoothing (15 points, second order) on normalized spectra. Savitsky-Golay smoothing is fully described in “Smoothing and Differentiation of Data by Simplified Least Squares Procedures”, Analytical Chemistry 1964;36;1627-1639, which is herein incorporated by reference in its entirety

### Results

### Histological Structures as Revealed by IR Micro-imaging

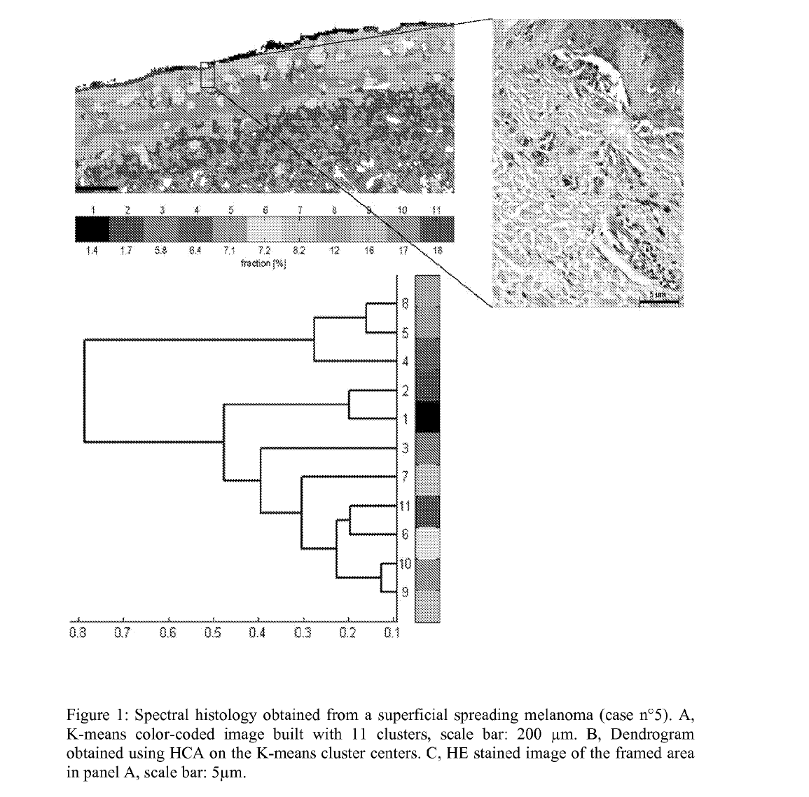

In each colour-coded image, pixels assigned to the same cluster represent an area of similar biochemical composition. Note that clustering was performed for each sample separately; no correspondence in the colours from one image to another exists. On the ten melanoma samples analyzed, a one-to-one correlation from those highly-contrasted images to the adjacent corresponding HE section was possible. A precise assignment of the image clusters to the tissular structures visible on the HE stained sections was thus feasible. It was possible to recover very detailed histological structures such as blood vessels and lymphocytes. In all cases, tumour areas were very well delineated and demarcated from the epidermis. For example, FIG. 1A depicts the colour-coded image obtained from a superficial spreading melanoma (case n°5) with a low Breslow thickness (0.35 mm) In this example, tumour cells (cluster 6, yellow) were located in the epidermal pegs but also in the invasive component. The stratum corneum (cluster 2, purple) and normal epidermis (cluster 7, pink) appear also very clearly. In addition, in each sample analyzed, IR spectra from the tumour and those from the epidermis were clearly bearing different biochemical profiles, as revealed by unsupervised HCA. In fact, this clustering analysis was performed on the cluster centres (average IR spectra of K-means clusters) of each pseudo-colour image to assess the spectral difference between the K-means clusters. The colour code of the dendrogram corresponds to the colour code used in the K-means image. The dendrogram obtained on the IR spectra from FIG. 1A is shown in FIG. 1C. It shows that cluster 7 is quite different (in terms of spectral distance) to cluster 6.

### Correlation Between IR Clusters and Dermatopathological Parameters

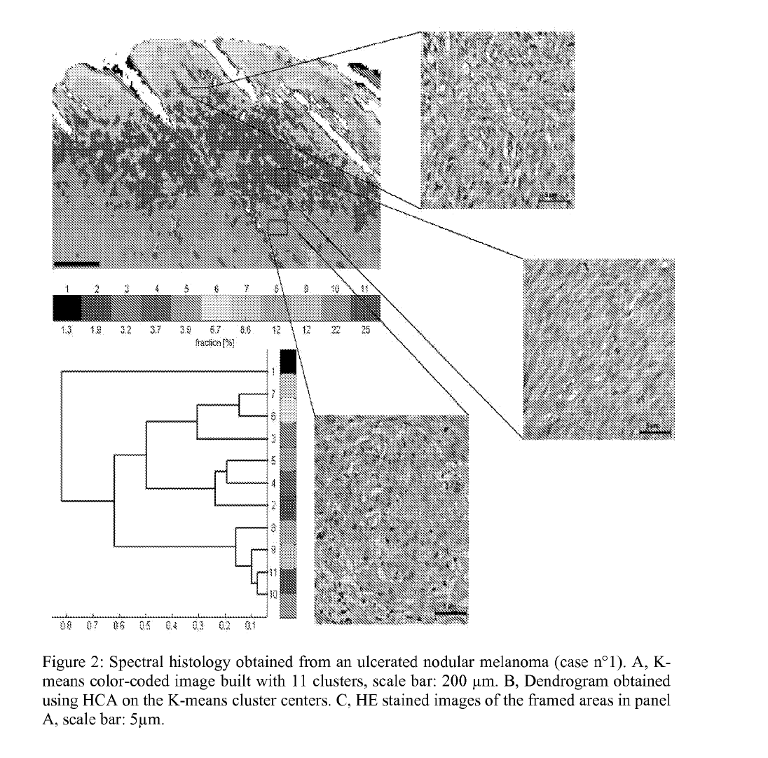

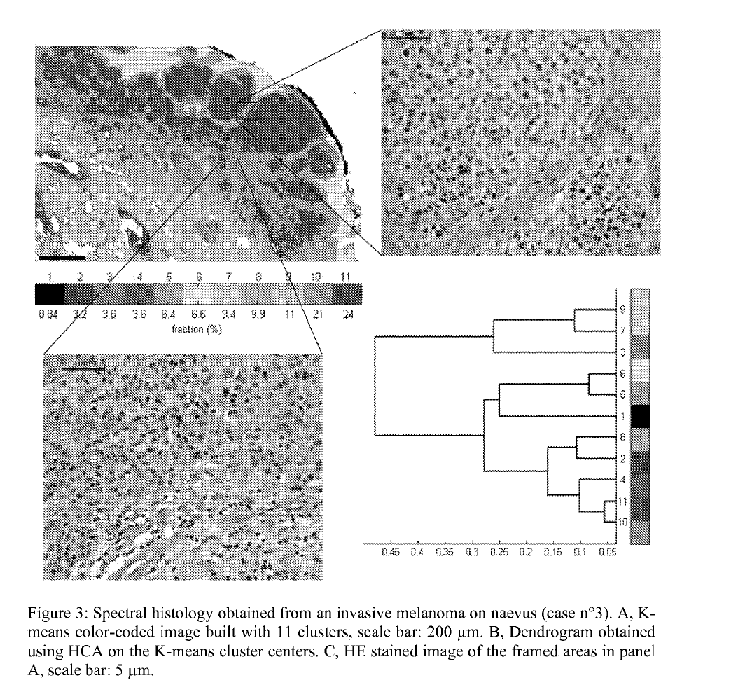

A significant association was observed between the presence of different tumour clusters and some dermatopathological parameters, in particular ulceration, Breslow thickness, Clark's level and number of mitoses per mm² Interestingly, only one tumour cluster was observed for good prognosis melanomas (3 cases) whereas 2 or 3 different tumour clusters were simultaneously present for bad prognosis melanoma (7 cases). No difference in the number of cluster was found between tumours with and without an adjacent naevus. FIG. 2A corresponds to the colour-coded K-means image obtained from an ulcerated nodular melanoma (case n°1). In this sample, three clusters (9-10-11) were assigned to tumoral melanocytes. The HCA (FIG. 2C) confirms that these three clusters carried similar biomolecular profiles. FIG. 3A shows the spectral analysis performed on a section from an invasive melanoma developed on a pre-existing nevus (case n°3). Tumour cells, assigned here to two clusters (10 and 11), were well demarcated from the basal layer (cluster 5) and the mucosal layer (cluster 6) of the epidermis. Some residues of the adjacent benign naevus (cluster 8) remained in close contact to the tumour. This phenomenon was also observed in the dendrogram (FIG. 3C).

A closer examination of the HE stained sections at a higher magnification permitted to associate each different tumour cluster to a specific morphological characteristic of the tumour cells. In the ulcerated nodular melanoma (case n°1, FIG. 2A), cluster 9 corresponded to the superficial region of the tumour, composed of epithelioid tumour cells, a high number of mitoses per mm² and a high heterogeneity in cell morphology. Cluster 11 was composed of spindle-shaped melanocytic cells, cellular density was higher and anisokaryosis less significant than in the neighbouring cluster 9. As the tumour infiltrated the surrounding stroma, tumour cells (cluster 10) seemed to cluster into small groups and lost their spindle shape, in addition, the number of mitoses per mm² decreased. Similar correlations between the different tumour clusters and cellular morphology were found for the other samples analyzed in this study.

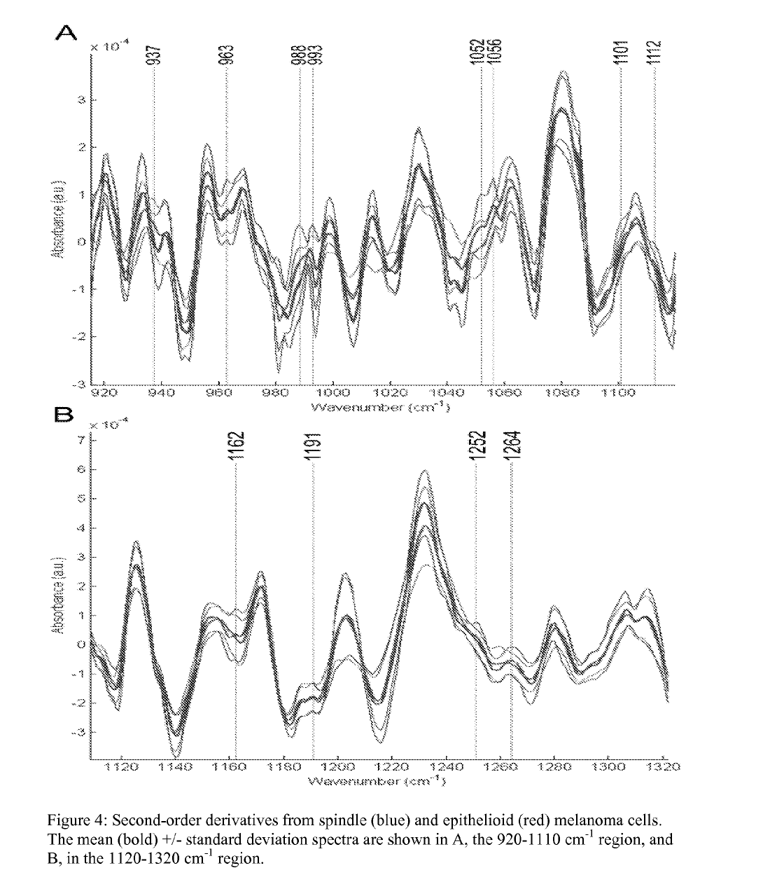

In order to investigate the molecular differences between the epithelioid and spindle morphologies, we compared their corresponding spectral signatures. For this purpose, the cluster centres associated to these cell morphologies were extracted from each K-means image. The resulting mean and standard deviation spectra were plotted to assess the significance of the differences observed (FIG. 4). Second-order derivatives were computed in order to enhance spectral differences. Table 2 lists the IR bands were major differences were observed, together with tentative band assignments (Baker M J, Gazi E, Brown M D, Shanks J H, Gardner P, Clarke N W. FTIR-based spectroscopic analysis in the identification of clinically aggressive prostate cancer. Br J Cancer 2008;99;1859-1866). Main spectral differences were attributed to DNA and RNA vibrations. Some modifications of protein content and protein conformation were also revealed (Amide III vibrations). Taken together these results, it seemed that the biomolecular differences between the two types of melanoma cells corresponded mainly to DNA and RNA. These differences between the distinctive tumoral structures as identified by IR micro-imaging could reflect modifications of relative concentration of DNA and RNA and/or differences of degrees of phosphorylation.

TABLE 2
Main spectral differences observed between
spindle and epithelioid melanoma cells

| Band position (cm⁻¹) | Assignment |
|---|---|
| 937 | |
| 963 | PO₄⁻ symmetric stretching |
| | Deoxyribose phosphate Skeletal motions |
| 988 | RNA stretching, ring bending of uracil |
| 993 | RNA stretching, ring bending of uracil |
| 1052 | C—O stretching in deoxyribose, ribose, DNA and |
| | RNA |
| 1056 | C—O stretching and bending of C—OH groups |
| | of carbohydrates |
| 1101 | |
| 1112 | Phosphate groups in DNA and RNA |
| 1162 | Phosphate groups in DNA and RNA |
| 1191 | Amide III vibrations (proteins) |
| 1252 | Amide III vibrations (proteins) |
| 1264 | Amide III vibrations (proteins) |

### Tissue Organization as Revealed by Spectral Analysis

The analysis of dendrograms obtained by unsupervised HCA analysis highlighted tissue organization based on its biochemical constitution. Tissular structures which spectral clusters are close in the dendrogram may not be necessarily in close contact but do reflect similar biomolecular information. For example, blood vessels, lymphocytes and erythrocytes dispersed within a tissue section were generally grouped in the same HCA cluster and all play crucial roles in the inflammatory reaction process. Spectra as defined by the pathologist as corresponding to peritumoral stroma or intratumoral stroma were closely associated to those of the tumour, as shown in FIG. 1. In this example, the dense stroma in the peritumoral dermis (clusters 9-10) and lymphocytes (cluster 11) were grouped in the same HCA cluster as the tumour cells (cluster 6). In addition, blood vessels (clusters 5-4) and melanophages (cluster 8) were also clustered in the same group. In only one case, the tumour cluster was spectrally closed to the one of the mucosal layer of the epidermis. These spectra corresponded to a tumoral area located in the intra-epidermal region of case n°7.

### Discussion

To date, the gold standard for the diagnosis of primary cutaneous melanoma is based on the histopathological diagnosis. The ability of a clinician to make a correct diagnosis is critical since patient life may depend on how early melanoma is diagnosed. Many attempts have been made to develop imaging techniques as reliable methods to allow differentiating between clinical suspected tumours. Thus, the identification of specific IR spectroscopic markers for the early diagnosis of lesions of poor prognosis would help optimize the therapeutic strategy.

The purpose of this proof-of-concept study was to demonstrate the potential of FTIR imaging for providing additional prognostic values to patients with primary cutaneous melanoma. The combination of FTIR imaging and multivariate statistical analyses has been previously used on other tissue types to reproduce histology. ((See Lasch P, Haensch W, Naumann D, Diem M., “imaging of colorectal adenocarcinoma using FT-IR microspectroscopy and cluster analysis”, Biochim Biophys Acta 2004;1688;176-186; Ly E, Piot O, Wolthuis R, Durlach A, Bernard P, Manfait M., “combination of FTIR spectral imaging and chemometrics for tumour detection from paraffin-embedded biopsies”, Analyst 2008;133;197-205; and Wolthuis R, Travo A, Nicolet C., “IR spectral imaging for histopathological characterization of xenografted human colon carcinomas”, Anal Chem 2008;80;8461-8469). As we surprisingly had performed a precise association between IR spectra and tissue structures, we built a spectral library helpful to identify spectral markers of each tissue type. Then, we developed an automatic predictive diagnostic algorithms, using pattern recognition techniques such as linear discriminant analysis and artificial neural networks, for an objective diagnosis of tissue samples. This methodology has been recently proved to be very efficient by our group for the differential diagnosis and characterization of skin carcinomas (basal cell carcinoma, squamous cell carcinoma and Bowen's disease). To the best of our knowledge, this is the first report on the FTIR imaging analysis of a collective series of primary cutaneous melanoma including different histological subtypes.

Among the primary cutaneous melanoma included in our study sample, one had a Clark level I (10%), two were level II (20%), one was level III (10%) and six were level IV (60%). As reported previously, FTIR imaging was able to identify spectroscopic markers of melanoma cells compared to normal epidermis. The highly contrasted colour-coded images generated in our study clearly separate melanoma cells from epidermis and benign adjacent naevus. In addition, an interesting finding from our survey was that the different tumour clusters revealed by FTIR imaging were associated with differences in cell morphology. On histological examination, this heterogeneity appeared very marked in nodular melanoma or in melanoma associated with a high level of invasion. In these cases, melanoma cells were packed and presented an epithelioid or spindle form. Melanoma cells can present different phenotypes, and the presence of different tumour cell morphologies in the same biopsy has been correlated to the metastatic risk. (See “Pathological findings suggestive of interclonal stabilization in a case of cutaneous melanoma”, Clin Exp Metastasis 1996; 14; 215-218). Likewise, in the biopsies analyzed in this study, tumour heterogeneity was only detected in bad prognosis melanomas. As such, the combination of IR imaging and pattern recognition techniques might be an innovative, label-free, rapid and automatic technology to screen high-risk metastatic melanoma patients.

Interestingly, in these same cases, the clusters obtained by FTIR imaging were associated with dominant dermatopathological parameters of poor prognosis, such as melanoma thickness, ulceration, level of invasion and mitotic rate. We found no difference between tumours with and without an adjacent naevus. These prediction algorithms will permit to identify tumoral zones automatically, based on the spectral data bank gathered from the analysis of a large number of samples.

Moreover, the interactions between melanoma tumours and their surrounding stromas have been widely studied as they may also reflect the invasive potential of the tumour cells. The cluster images provide with highly contrasted colour images that make it possible to identify easily different types of stroma reactions. Indeed, the peritumoral stroma and the intratumoral stroma could be clearly distinguished from the normal stroma reaction. HCA revealed that these peritumoral and intratumoral stroma reactions carry similar IR bio-profiles related to those of melanoma cells. This analysis confirms the hypothesis that strong interactions exist between the tumour cells and their matrix environment. Remodelling of the stroma at the neighbourhood of the tumour is related to first, a high aggregation of inflammatory cells (e.g. lymphocytes, fibroblasts and macrophages), and second, to changes in vascular structures. In fact, in melanoma, blood vessels predominantly surround a closely packed neoplastic proliferation, whereas in normal dermis, the vascular network is widely distributed.

### CONCLUSION

FTIR imaging has promising applications in the diagnosis of primary cutaneous melanoma. Direct analysis of paraffin-embedded sections can be performed without any solvent-based removal of paraffin, and multivariate statistical analyses provide with highly contrasted colour-coded images that can reproduce tissue histology automatically. Good correlation between IR spectral clusters and dermatopathological parameters was feasible, suggesting that this rapid and automated procedure could help in improving and optimizing the diagnosis of primary cutaneous melanoma, and also provide with additional prognostic markers. The integration of this method to conventional laboratory procedures is feasible and could help in the guidance of the surgical procedure. As infrared imaging systems are becoming more and more efficient, it is now possible to record a spectral image from a large tissue sample within a matter of minutes, making it possible to pose a diagnosis in less than hour after biopsy.

1. A process for identifying and classifying cutaneous melanoma comprising: a) scanning a lesion on the skin of a subject by a FTIR spectrometer coupled with a micro-imaging system; b) acquiring and storing infrared spectra of a series of digital images of the lesion; c) clustering the infrared spectra by a statistics based clustering algorithm, such as a multivariate statistical analyzer using a K-means classification algorithm; d) comparing the cluster-membership information to a spectral library of various tissue types of cutaneous melanoma to identify spectral markers of each tissue type of the cutaneous melanoma; and e) mapping the spectral markers by assigning a color to each different cluster.

2. A process according to claim 1, wherein the infrared spectra of the lesion are real tissue-specific spectroscopic fingerprints.

3. A process according to claim 1, wherein the color-coded mapping highlights tissue architecture without staining.

4. A process according to claim 1, wherein the process discriminates tumor areas from normal epidermis.

5. A process according to claim 1, wherein the process differentiates histological subtypes of the cutaneous melanoma.

6. A process according to claim 1, wherein each digital image consists of from about 10,000 to about 100,000 spectra.

7. A process according to claim 6, wherein each digital image consists of about 30,000 spectra.

8. A process according to claim 7, wherein each spectrum contains from 1,000 to 50,000 values of absorbance.

9. A process according to claim 8, wherein each absorbance is from 720 to 4000 cm⁻¹.

10. A process according to claim 9, wherein each absorbance is from 900 to 1800 cm⁻¹.

11. A process according to claim 1, wherein the centers of the K-means cluster are normalized across the entire skinned area and a second-order derivative is calculated using Savitsky-Golay smoothing on the normalized spectra.

12. A process according to claim 1, wherein the process identifies different types of stroma reactions by clustering the infrared spectra.

13. A process according to claim 12, wherein peritumoral stroma and intratumoral stroma are distinguished from the normal stroma reaction.
